(19) United States
(12) Patent Application Publication
(10) Pub. No.: US 2024/0255516 A1
(43) Pub. Date: Aug. 1, 2024

(54) IN VITRO METHOD OF PREDICTING RUMEN DIGESTIBLE PROTEIN

(71) Applicant: CAN TECHNOLOGIES, INC., Wayzata, MN (US)

(72) Inventors: Jeremy Chignell, Northfield, MN (US); Cathy Staloch Hass, Plymouth, MN (US); Gladys Ethel Margaria, Princeton, MN (US); Gregory Michael Poynter, Saint Paul, MN (US); Michael Telander, Otsego, MN (US); Stephanie Zebro, Zimmerman, MN (US); Alessandro Zontini, Casalmaggiore (IT)

(73) Assignee: CAN TECHNOLOGIES, INC., Wayzata, MN (US)

(21) Appl. No.: 18/561,078

(22) PCT Filed: May 12, 2022
(86) PCT No.: PCT/US2022/072288

§ 371 Date: Nov. 15, 2023

Related U.S. Application Data
(60) Provisional application No. 63/194,393, filed on May 28, 2021.

(51) Int. Cl. G01N 33/68

(52) U.S. Cl. CPC G01N 33/6803; G01N 2333/962

(57) ABSTRACT

The present disclosure relates to in vitro methods of predicting rumen digestible protein of a feed sample. An in vitro method of predicting rumen digestible protein of a feed sample includes forming an aqueous test composition including the feed sample, a buffer, and one or more digestive enzymes. The method includes incubating the test composition. The method includes removing one or more test samples of the test composition at time points during the incubating, the time points including about 0 h and at least one time point after about 0 h. The method includes measuring soluble protein content of the one or more test samples. The method includes using the measured soluble protein content of the one or more test samples to predict a rate and/or extent of rumen protein digestion of the feed sample.

### CROSS-REFERENCE TO RELATED APPLICATIONS

This application claims the benefit of U.S. Provisional Patent Application No. 63/194,393, filed May 28, 2021, which is incorporated by reference herein in its entirety.

### TECHNICAL FIELD

This application relates to methods for determining rumen digestible protein content of a feed sample.

### BACKGROUND

Many feeding systems for ruminants now require knowledge of rumen digestible protein content. Rumen digestible protein is the portion of total protein that is degraded in the rumen. Currently, the in situ procedure is commonly used to estimate rumen digestible protein concentrations in ruminant feedstuffs; however, this procedure has technical limitations, is difficult to standardize, and requires ruminal fistulated animals that are expensive to maintain and frequently unavailable to commercial laboratories. In vitro methods that measure the overall percent of rumen un-digestible protein (the portion of total protein that is not degraded in the rumen) in a feed sample cannot be used to accurately predict dynamic parameters such as rate of protein digestion and rate of feed passage.

Accordingly, there is an ever-growing need to have improved methods for determining rumen digestible protein content in addition to dynamic parameters such as rate of protein digestion and rate of feed passage as described herein.

### SUMMARY OF THE INVENTION

Various aspects of the present invention provide an in vitro method of predicting rumen digestible protein of a feed sample. The method includes forming an aqueous test composition. The aqueous test composition includes the feed sample, a buffer, and one or more digestive enzymes. The method includes incubating the test composition. The method includes removing one or more test samples of the test composition at time points during the incubating. The time points at which the samples are removed includes about 0 h and at least one time point after about 0 h. The method includes measuring soluble protein content of the one or more test samples. The method also includes using the measured soluble protein content of the one or more test samples to predict a rate and/or extent of rumen protein digestion of the feed sample.

Various aspects of the present invention provide an in vitro method of predicting rumen digestible protein of a feed sample. The method includes forming an aqueous test composition. The aqueous test composition includes the feed sample, a buffer having a pH of about 5 to about 9, and one or more digestive enzymes. Forming the test composition includes combining the feed sample and the buffer to form a pre-test composition. Forming the test composition also includes pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition. The method includes incubating the test composition at a temperature of about 35° C. to about 45° C. with agitation at about 50 RPM to about 150 RPM. The method includes removing one or more test samples of the test composition at time points during the incubating, the time points including about 0 h and at least one time point after about 0 h. The method includes measuring soluble protein content of the one or more test samples. The method also includes using the measured soluble protein content of the one or more test samples to predict a rate and/or extent of rumen protein digestion of the feed sample.

Various aspects of the present invention provide an in vitro method of predicting rumen digestible protein of a feed sample. The method includes forming an aqueous test composition. The aqueous test composition includes the feed sample, wherein the feed sample includes soybeans and/or rapeseed. The aqueous test composition also includes a buffer having a pH of about 5.8 to about 7.2 and one or more digestive enzymes. Forming the test composition includes combining the feed sample and the buffer to form a pre-test composition, and pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition. The one or more digestive enzymes includes one or more proteases from Aspergillus oryzae having an activity of about 0.1 U/mL to about 1.5 U/mL in the test composition per about 0.2 g of protein in the feed sample. The method includes incubating the test composition at a temperature of about 35° C. to about 45° C. with agitation at about 50 RPM to about 150 RPM. The method includes removing one or more test samples of the test composition at time points during the incubating, the time points including about 0 h and at least one time point after about 0 h. The method includes measuring soluble protein content of the one or more test samples. The method also includes using the measured soluble protein content of the one or more test samples to predict a rate and/or extent of rumen protein digestion of the feed sample.

Various aspects of the present invention provide an in vitro method of predicting rumen digestible protein of a feed sample. The method includes forming an aqueous test composition including the feed sample. The feed sample includes distillers grains. The aqueous test composition also includes a buffer having a pH of about 5.8 to about 7.2 and one or more digestive enzymes. Forming the test composition includes combining the feed sample and the buffer to form a pre-test composition, and pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition. The one or more digestive enzymes include pancreatin from porcine pancreas having a concentration of about 0.01 mg/mL to about 0.15 mg/mL in the test composition per about 0.2 g of protein in the feed sample. The method includes incubating the test composition at a temperature of about 35° C. to about 45° C. with agitation at about 50 RPM to about 150 RPM. The method includes removing one or more test samples of the test composition at time points during the incubating, the time points including about 0 h and at least one time point after about 0 h. the method includes measuring soluble protein content of the one or more test samples. The method also includes using the measured soluble protein content of the one or more test samples to predict a rate and/or extent of rumen protein digestion of the feed sample.

Various aspects of the present invention provide an in vitro method for prediction of the rate and/or extent of rumen digestible protein content of a feed sample rather than only the overall percent of rumen un-digestible protein in a feed sample. Various aspects of the present invention allow ruminant nutritionists to more accurately predict rumen digestible protein of a feed sample (e.g., by taking advantage of dynamic parameters such as rate of protein digestion and rate of feed passage), and/or to increase or maximize the efficiency of nitrogen usage. In various aspects, the in vitro method of the present invention is less expensive and/or provides more consistent results than using an in situ method (i.e., with fistulated animals) and/or a rumen fluid fermentation process. In various aspects, the in vitro method of the present invention provides faster throughput of samples, enabling more feed samples to be analyzed in less time, as compared to an in situ method and/or rumen fluid fermentation process. In various aspects, the in vitro method of the present invention, the prediction of the rate and/or extent of rumen digestible protein content of a feed sample is highly correlated with an in situ method.

### BRIEF DESCRIPTION OF THE FIGURES

The drawings illustrate generally, by way of example, but not by way of limitation, various aspects of the present invention.

FIG. 1A illustrates percent soluble protein versus time for feed sample SBM 47, in accordance with various aspects.

FIG. 1B illustrates percent soluble protein versus time for a soybean-containing feed sample, in accordance with various aspects.

FIG. 1C illustrates percent soluble protein versus time for a rapeseed-containing feed sample, in accordance with various aspects.

FIG. 1D illustrates percent soluble protein versus time for a rapeseed-goldrape feed sample, in accordance with various aspects.

FIG. 2A illustrates percent soluble protein versus time for feed sample SBM 47, in accordance with various aspects.

FIG. 2B illustrates percent soluble protein versus time for a soybean-containing feed sample, in accordance with various aspects.

FIG. 3A illustrates percent soluble protein versus time for a DDGS feed sample, in accordance with various aspects.

FIG. 3B illustrates percent soluble protein versus time for a mix DDGS feed sample, in accordance with various aspects.

FIG. 4A illustrates a plot of the proportion of remaining crude protein in the residue versus time for SBM47 in Example 1, in accordance with various aspects.

FIG. 4B illustrates a plot of the proportion of remaining crude protein in the residue versus time for soypass in Example 1, in accordance with various aspects.

FIG. 4C illustrates a plot of the proportion of remaining crude protein in the residue versus time for rapeseed meal in Example 1, in accordance with various aspects.

FIG. 4D illustrates a plot of the proportion of remaining crude protein in the residue versus time for goldrape in Example 1, in accordance with various aspects.

FIG. 4E illustrates a plot of the proportion of remaining crude protein in the residue versus time for DDGS in Example 2, in accordance with various aspects.

FIG. 4F illustrates a comparison of rates for SBM, soypass, rapeseed, goldrape, from Example 1, and DDGS, from Example 2, in accordance with various aspects.

FIG. 5A illustrates percent soluble protein versus time as determined via the total nitrogen analysis for feed samples Legume Hay, from Example 5, in accordance with various aspects.

FIG. 5B illustrates percent soluble protein versus time as determined via the total nitrogen analysis for feed sample alfalfa haylage, from Example 5, in accordance with various aspects.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Method of Predicting Rumen Digestible Protein of a Feed Sample.

Various aspects of the present invention provide a method of predicting rumen digestible protein of a feed sample. The method includes forming an aqueous test composition. The aqueous test composition can include the feed sample, a buffer, and one or more digestive enzymes. The method can include incubating the test composition. The method can include removing one or more test samples of the test composition at time points during the incubating. The time points include about 0 h and also include at least one time point after about 0 h. The method can include measuring soluble protein content of the one or more test samples. The method can also include using the measured soluble protein content of the one or more test samples to predict a rate and/or extent of rumen protein digestion of the feed sample.

The aqueous test composition can include the feed sample. The feed sample can be any suitable feed sample for consumption by animals, such as alone or as a mixture with other ingredients. The feed sample can include an animal feed, a feedstuff, or a combination thereof. An animal feed can include a complete feed, a premix, a concentrate, a base mix, a supplement, a top dress, or a combination thereof. The feed sample can include barley, blood meal, bone meal, Brewer's grain, corn grain, corn gluten meal, corn gluten feed, cottonseed (e.g., whole or meal), distiller's grain, fish meal, hominy, feather meal, molasses, peanut skins, soybeans (e.g., whole or meal), tallow, wheat (e.g., whole, bran or middlings), a rumen-protected amino acid, a byproduct thereof (e.g., any animal feedstuff made or derived from all or a portion of any one or combination of the foregoing), or a combination thereof. The feed sample can include forage, corn, wheat, soybean, oats, barley, beet pulp, citrus pulp, cottonseed, sunflowers, canola/rapeseed, rice, peas, rye, distillers grains, a byproduct thereof (e.g., any animal feedstuff made or derived from all or a portion of any one or combination of the foregoing), or a combination thereof. Forage, for example, may include Brown midrib corn silage, corn silage, ensiled barley, ensiled fresh legume, ensiled fresh mix forage, ensiled fresh small grain, ensiled grass (e.g., ensiled ryegrass, canary, and/or orchard grass), ensiled fresh grass, ensiled legume (e.g., ensiled alfalfa), ensiled mix forage (e.g., ensiled forages that include mixes of legumes, grasses, small grains; e.g., ensiled alfalfa/grass mixture), ensiled small grain (e.g., ensiled rye, triticale, oats), ensiled sorghum (e.g., ensiled Sudan grass), grass hay (e.g., dry ryegrass, canary, or orchard grass), legume hay (e.g., dry alfalfa), mix forage hay (e.g., dry hay that includes mixes of legumes, grasses, and/or small grains, dry alfalfa, and grass mixed), small grain hay (e.g., dry rye, triticale, oats), or a combinations. The feed sample can include distillers grains, soybeans, rapeseed, a byproduct thereof, or a combination thereof. The feed sample can include soybeans, rapeseed, byproducts thereof, or a combination thereof. The feed sample can include distillers grains, byproducts thereof, or a combination thereof. As used herein, “distillers grains” includes dried distillers grains (DDG), distillers dry grains with solubles (DDGS), distillers wet grains (DWG), and distillers solubles (DS).

In some aspects, forming the test composition can include combining the feed sample and the buffer to form a pre-test composition. The method can include pre-incubating the pre-test composition prior to adding the one or more digestive enzymes to form the aqueous test composition. The pre-incubating of the pre-test composition can include any suitable conditions. The pre-incubating can include subjecting the pre-test composition to agitation, elevated temperature, or a combination thereof. The agitation can be any suitable agitation, such as shaking or stirring. The agitation can include agitation at about 20 RPM to about 1000 RPM, about 50 RPM to about 150 RPM, about 80 RPM to about 100 RPM, or less than or equal to 1000 RPM but greater than or equal to 20 RPM, 30, 40, 50, 60, 70, 75, 80, 82, 84, 86, 88, 90, 92, 94, 96, 98, 100, 105, 110, 120, 130, 140, 150, 160, 170, 180, 190, 200, 250, 500, or 750 RPM. The temperature of the preincubation can be any suitable temperature, such as a temperature of about 35° C. to about 45° C., about 38° C. to about 40° C., about 39° C., or equal to or less than about 45° C. and greater than or equal to about 35° C., 36, 37, 38, 39, 40, 41, 42, 43, or 44° C. The preincubation can be performed for any suitable duration, such as for about 30 minutes to about 2 hours, or about 45 minutes to about 1.5 hours, or about 1 hour, or less than or equal to 24 hours and greater than or equal to 1 minute, 2, 3, 4, 5, 10, 15, 20, 30, 40, 50 minutes, 1 hour, 1.2, 1.4, 1.6, 1.8, 2, 2.2, 2.4, 2.6, 2.8, 3, 4, 5, 6, 8, 10, 12, 14, 16, 18, 20, or 22 h. The pre-incubating of the pre-test composition can include agitation at about 50 RPM to about 150 RPM at a temperature of about 35° C. to about 45° C. for about 30 minutes to about 2 hours. The pre-incubating of the pre-test composition can include agitation at a temperature of about 38° C. to about 40° C. for about 45 minutes to about 1.5 hours. The pre-incubating of the pre-test composition can include agitation at a temperature of about 39° C. for about 1 hour.

The buffer can have any suitable pH, such as a pH of about 5 to about 9, about 5.8 to about 7.2, about 6.7, or less than or equal to 9 and greater than or equal to 5, 5.2, 5.4, 5.6, 5.8, 6, 6.1, 6.2, 6.3, 6.4, 6.5, 6.6, 6.7, 6.8, 6.9, 7, 7.1, 7.2, 7.4, 7.6, 7.8, 8, 8.2, 8.4, 8.6, or 8.8. The test composition can have any suitable pH, such as a pH of about 5 to about 9, about 5.8 to about 7.2, about 6.7, or less than or equal to 9 and greater than or equal to 5, 5.2, 5.4, 5.6, 5.8, 6, 6.1, 6.2, 6.3, 6.4, 6.5, 6.6, 6.7, 6.8, 6.9, 7, 7.1, 7.2, 7.4, 7.6, 7.8, 8, 8.2, 8.4, 8.6, or 8.8. The buffer can be any suitable buffer. For example, the buffer can include citric acid, acetic acid, KH₂PO₄, K₂HPO₄, K₃PO₄, HCl, KCl, N-cyclohexyl-2-aminoethanesulfonic acid (CHES), glycine, potassium hydrogen phthalate, tetraboric acid, boric acid, [tris(hydroxymethyl)methylamino]propanesulfonic acid (TAPS), 2-(bis(2-hydroxyethyl)amino)acetic acid (bicine), tris(hydroxylmethyl)aminomethane (tris), N-[tris(hydroxymethyl)methyl]glycine (tricine), 3-[N-tris(hydroxymethyl)methylamino]-2-hydroxypropanesulfonic acid (TAPSO), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), piperazine-N,N′-bis(2-ethanesulfonic acid) (PIPES), dimethylarsenic acid (cacodylate), 2-(N-morpholino)ethanesulfonic acid (MES), a salt thereof, borate, sodium hydroxide, potassium hydroxide, sodium tetraborate, sodium carbonate, potassium carbonate, potassium chloride, sodium chloride, a sodium phosphate, phosphate buffered saline (PBS), hydrates thereof, or a combination thereof. The buffer can include a sodium phosphate, phosphate buffered saline (PBS), a borate-phosphate buffer, or a combination thereof. The buffer can include monobasic sodium dihydrogen orthophosphate and di-sodium tetraborate decahydrate.

The one or more digestive enzymes can be any suitable enzymes that allow prediction of a rate and/or extent of rumen protein digestion of the feed sample using the method described herein. As used herein, “digestive enzyme” refers to an enzyme that breaks down molecules from food (e.g., polymeric macromolecules) into their smaller building blocks in order to facilitate their absorption by the body. The one or more digestive enzymes can include a protease, a metalloprotease, a lyase, an amylase, or a combination thereof. The one or more digestive enzymes can include a bacterial protease, an animal protease, a virus protease, a plant protease, a fungal protease, a serine protease, a cysteine protease, a threonine protease, an aspartic protease, a glutamic protease, a metalloprotease, an asparagine peptide lyase, an amylase derives from a pancreas, or a combination thereof. The one or more digestive enzymes can include a mammalian (e.g., bovine, human, or other) trypsin, trypsin from porcine pancreas, one or more Aspergillus-derived digestive enzymes, one or more proteases from Aspergillus oryzae, pancreatin from porcine pancreas, an amylase derived from a porcine pancreas, a protease derived from Bacillus licheniformis, a protease derived from Bacillus amyloliquefaciens, or a combination thereof. The one or more digestive enzymes can include one or more proteases from Aspergillus oryzae, pancreatin from porcine pancreas, or a combination thereof.

The type of the one or more digestive enzymes in the test composition, and the concentration of the one or more digestive enzymes in the test composition, can be sufficient such that a rate of formation of solubilized protein in the test composition during the incubation is within about 30%, 20%, 10%, or 5% of a rate of protein digestion of the feed sample in a rumen stomach (e.g., about 0% to about 30%, or about 5% to about 20%, or about 5% to about 10%, or less than or equal to 30% and greater than or equal to about 1%, 2, 3, 4, 5, 6, 7, 8, 9, 10, 11, 12, 13, 14, 15, 16, 17, 18, 19, 20, 22, 24, 26, or 28%). The type of the one or more digestive enzymes in the test composition, and the concentration of the one or more digestive enzymes in the test composition, can be sufficient such that an extent of formation of solubilized protein in the test composition during the incubation is within about 30%, 20%, 10%, or 5% of an extent of protein digestion of the feed sample in a rumen stomach (e.g., about 0% to about 30%, or about 5% to about 20%, or about 5% to about 10%, or less than or equal to 30% and greater than or equal to about 1%, 2, 3, 4, 5, 6, 7, 8, 9, 10, 11, 12, 13, 14, 15, 16, 17, 18, 19, 20, 22, 24, 26, or 28%). The type and concentration of the one or more digestive enzymes can be sufficient such that both the rate and the extent of formation of solubilized protein in the test composition during the incubation is within about 30%, 20%, 10%, or 5% of a rate and extent of protein digestion of the feed sample in a rumen stomach (e.g., about 0% to about 30%, or about 5% to about 20%, or about 5% to about 10%, or less than or equal to 30% and greater than or equal to about 0%, 1, 2, 3, 4, 5, 6, 7, 8, 9, 10, 11, 12, 13, 14, 15, 16, 17, 18, 19, 20, 22, 24, 26, or 28%). The rate and extent of protein digestion of the feed sample in the rumen stomach can be measured via an in vivo method using a fistulated ruminant animal.

The type of the one or more digestive enzymes in the test composition, and the concentration of the one or more digestive enzymes in the test composition, can be sufficient such that a rate of formation of solubilized protein in the test composition during the incubation is within about 3, 2, or 1 standard deviations of a rate of protein digestion of the feed sample in a rumen stomach (e.g., about 0 to about 3, or about 0.1 to about 2, or about 0.1 to about 1, or less than or equal to about 3 and greater than or equal to about 0, 0.2, 0.4, 0.6, 0.8, 1, 1.2, 1.4, 1.6, 1.8, 2, 2.2, 2.4, 2.6, or 2.8). The type of the one or more digestive enzymes in the test composition, and the concentration of the one or more digestive enzymes in the test composition, can be sufficient such that an extent of formation of solubilized protein in the test composition during the incubation is within about 3, 2, or 1 standard deviations of an extent of protein digestion of the feed sample in a rumen stomach (e.g., about 0 to about 3, or about 0.1 to about 2, or about 0.1 to about 1, or less than or equal to 3 and greater than or equal to about 0, 0.2, 0.4, 0.6, 0.8, 1, 1.2, 1.4, 1.6, 1.8, 2, 2.2, 2.4, 2.6, or 2.8). The type and concentration of the one or more digestive enzymes can be sufficient such that both the rate and the extent of formation of solubilized protein in the test composition during the incubation is within about 3, 2, or 1 standard deviations of a rate and extent of protein digestion of the feed sample in a rumen stomach (e.g., about 0 to about 3, or about 0.1 to about 2, or about 0.1 to about 1, or less than or equal to 3 and greater than or equal to about 0, 0.2, 0.4, 0.6, 0.8, 1, 1.2, 1.4, 1.6, 1.8, 2, 2.2, 2.4, 2.6, or 2.8). The rate and extent of protein digestion of the feed sample in the rumen stomach can be measured via an in vivo method using a fistulated ruminant animal.

The concentration of the one or more digestive enzymes in the test composition can be sufficient such that they have an activity (e.g., after the test composition is formed but before the onset of the incubation) of about 0.001 U/mL to about 1000 U/mL per 0.2 g of protein in the feed sample, or about 0.01 U/mL to about 100 U/mL, or about 0.1 U/mL to about 10 U/mL, or less than or equal to 1000 U/mL and greater than or equal to 0.001 U/mL, 0.005, 0.01, 0.05, 0.1, 0.5, 1, 2, 3, 4, 5, 6, 8, 10, 12, 14, 16, 18, 20, 25, 50, 75, 100, 125, 150, 175, 200, 300, 400, 500, or 750 U/mL. The concentration of the one or more digestive enzymes in the test composition can be about 0.0001 mg/mL to about 500 mg/mL per 0.2 g of protein in the feed sample, or about 0.001 mg/mL to about 100 mg/mL, or about 0.01 mg/mL to about 10 mg/mL, or less than or equal to 500 mg/mL and greater than or equal to 0.0001 mg/mL, 0.0005, 0.001, 0.005, 0.01, 0.05, 0.1, 0.5, 1, 2, 3, 4, 5, 6, 8, 10, 12, 14, 16, 18, 20, 25, 50, 75, 100, 125, 150, 175, 200, 300, or 400 mg/mL.

In various aspects, the one or more digestive enzymes can include one or more proteases from Aspergillus oryzae. The one or more proteases from Aspergillus oryzae can have an activity of about 0.1 U/mL to about 1.5 U/mL in the test composition per about 0.2 g of protein in the feed sample (e.g., after the test composition is formed but before the onset of the incubation), or about 0.4 U/mL to about 0.9 U/mL, or about 0.64 U/mL, or less than or equal to 1.5 U/mL and greater than or equal to about 0.1 U/mL, 0.2, 0.3, 0.4, 0.5, 0.6, 0.7, 0.8, 0.9, 1, 1.1, 1.2, 1.3, or 1.4 U/mL. The feed sample can include any suitable feed sample, such as including soybeans, rapeseed, byproducts thereof, or a combination thereof.

In various aspects, the one or more digestive enzymes can include a protease from porcine pancreas, such as more than one enzyme from porcine pancreas. The one or more enzymes from porcine pancreas can have a concentration of about 0.01 mg/mL to about 0.15 mg/mL in the test composition per about 0.2 g of protein in the feed sample, or about 0.04 mg/mL to about 0.09 mg/mL, or about 0.067 mg/mL, or less than or equal to about 0.15 mg/mL and greater than or equal to about 0.01 mg/mL, 0.02, 0.03, 0.04, 0.05, 0.06, 0.07, 0.08, 0.09, 0.10, 0.11, 0.12, 0.13, or 0.14 mg/mL. The feed sample can include any suitable feed sample, such as including distillers grains, byproducts thereof, or a combination thereof.

In some aspects, the type of enzymes and the desired concentrations thereof in the test composition are known prior to the onset of the method. In some aspects, the method can include determining the type of enzymes and the desired concentrations thereof in the test composition. For example, in various aspects, the method can further includes pre-determining an amount or activity level of the one or more digestive enzymes in the test composition for the feed sample. The pre-determining of the amount or the activity level of the one or more digestive enzymes in the test composition can include optimizing an amount of the one or more digestive enzymes used in the test composition such that a rate and/or extent of formation of solubilized protein in the test composition during the incubation is within about 3, 2, or 1 standard deviations of a rate and/or extent of protein digestion of the feed sample in a rumen stomach (e.g., about 0 to about 3, or about 0.1 to about 2, or about 0.1 to about 1, or less than or equal to about 3 and greater than or equal to about 0, 0.2, 0.4, 0.6, 0.8, 1, 1.2, 1.4, 1.6, 1.8, 2, 2.2, 2.4, 2.6, or 2.8). The pre-determining of the amount or the activity level of the one or more digestive enzymes in the test composition can include optimizing an amount of the one or more digestive enzymes used in the test composition such that a rate and/or extent of formation of solubilized protein in the test composition during the incubation is within 30%, 20%, 10%, or 5% of a rate and/or extent of protein digestion of the feed sample in a rumen stomach (e.g., about 0% to about 30%, or about 5% to about 20%, or about 5% to about 10%, or less than or equal to 30% and greater than or equal to about 0%, 1, 2, 3, 4, 5, 6, 7, 8, 9, 10, 11, 12, 13, 14, 15, 16, 17, 18, 19, 20, 22, 24, 26, or 28%).

The method can include incubating the test composition. The incubating can be performed under any suitable conditions that allow prediction of a rate and/or extent of rumen protein digestion of the feed sample using the method described herein. For example, the incubating can be performed at a temperature of about 30° C. to about 50° C., or about 35° C. to about 45° C., or about 38° C. to about 40° C., or about 39° C., or less than or equal to 50° C. and greater than or equal to about 30° C., 31, 32, 33, 34, 35, 36, 37, 38, 39, 40, 41, 42, 43, 44, 45, 46, 47, 48, or 49° C. The incubating can be performed for a duration of about 1 minute to about 1 week, about 30 minutes to about 96 hours, about 1 hour to about 72 hours, or less than or equal to 1 week and greater than or equal to about 1 minute, 2, 3, 4, 5, 6, 8, 10, 12, 14, 16, 18, 20, 25, 30, 35, 40, 45, 50, 55 minutes, 1 h, 1.5, 2, 2.5, 3, 3.5, 4, 4.5, 5, 6, 8, 10, 12, 14, 16, 18, 20, 22, 24, 36, 48, 60, 72, 84, 96 h, 5 d, or 6 d. The incubating can include agitating the test composition, such as by shaking or stirring the test composition. The agitating can include agitating at about 20 RPM to about 1000 RPM, about 50 RPM to about 150 RPM, about 80 RPM to about 100 RPM, or less than or equal to 1000 RPM but greater than or equal to 20 RPM, 30, 40, 50, 60, 70, 75, 80, 82, 84, 86, 88, 90, 92, 94, 96, 98, 100, 105, 110, 120, 130, 140, 150, 160, 170, 180, 190, 200, 250, 500, or 750 RPM. The incubating can include agitation at about 50 RPM to about 150 RPM at a temperature of about 35° C. to about 45° C. for about 1 hour to about 72 hours. The incubating can include agitation at about 80 RPM to about 100 RPM at about 39° C. for about 1 hour to about 72 hours.

The method can include removing one or more test samples of the test composition at time points during the incubating. The removing of the sample of the test composition can include removing a portion of supernatant solution from the test composition. The time points at which the sample of the test composition is removed during the incubating includes about 0 h, and also includes at least one other time point after 0 h, such as one time point after 0 h or more than one time point after 0 h (e.g., 2 time points after 0 h, 3, 4, 5, 6, 7, 8, 9, 10, 12, 14, 16, 18, 20, or more). In some examples, the at least one time point after about 0 h at which the sample of the test composition is removed during the incubating includes one or more of about 4 h, 24, 48, and about 72 h.

The method can include measuring soluble protein content of the one or more test samples. As used herein, the term “soluble protein content” refers to the concentration of protein in samples of supernatant taken from the aqueous test composition. The measuring of the soluble protein content of the one or more test samples can be performed in any suitable way. Any of a variety of conventional techniques for measuring soluble protein content can be used in the present method. For example, the measuring of the soluble protein content of the one or more test samples can include a combustion method (e.g., on a LECO instrument), a digestion method, total nitrogen analysis, a microplate assay method, a cuvette assay method (e.g., using the same reagents as a microplate assay method), a 2,4,6-trinitrobenzene sulfonic acid (TNBS) assay, a bicinchoninic acid (BCA) assay, acid hydrolysis followed by amino acid measurement (e.g., via HPLC or free amine detection reagents such as ninhydrin or TNBS), amino acid HPLC, quantitative protein gel, microfluidic electrophoretic separation (e.g., using a LapChip system), Kjeldahl method, Lowry colorimetric method, or a combination thereof. Microfluidic electrophoretic separation can be used to measure protein concentration of larger peptides. The measuring of the soluble protein content can include total nitrogen analysis and/or a microplate assay method. The measuring of the soluble protein content can include total nitrogen analysis. The measuring of the soluble protein content can include a microplate assay method. A microplate assay method can include a protein concentration assay, an assay for detection of free amino groups, an assay for colorimetric detection of free amino groups, or a combination thereof. A microplate assay method can include a ninhydrin assay. The measuring of the soluble protein content can include combining a protein concentration determined via a protein concentration assay and a protein concentration determined via a ninhydrin assay to determine the measured soluble protein content of the sample.

The method can include using the measured soluble protein content of the one or more test samples to predict a rate of rumen protein digestion of the feed sample. The method can include using the measured soluble protein content of the one or more test samples to predict an extent of rumen protein digestion of the feed sample. The method can include using the measured soluble protein content of the one or more test samples to predict an extent and rate of rumen protein digestion of the feed sample. The using of the measured soluble protein content of the one or more test samples to predict an extent of rumen protein digestion of the feed sample can include subtracting the measured soluble protein content of the test composition at about 0 h from the measured soluble protein content of the test composition at the least one time point after about 0 h to predict the extent of rumen protein digestion of the feed sample at the at least one time point after about 0 h. The using of the measured soluble protein content of the one or more test samples to predict a rate of rumen protein digestion of the feed sample can include solving for kd in the equation:

$$soluble CP ( t ) = {soluble CP}^{( - kd * ( t - L ) )} unsoluble CP .$$

In this equation, solubleCP(t) is the protein content over time, kd is the predicted rate of rumen protein digestion of the feed sample, t is the time variable (e.g., a time the samples were removed), solubleCP is the measured soluble protein content of the removed sample at time t (e.g., the extent of soluble degradable protein at time t, extent of protein digestion at time t, or maximum soluble degradable protein at time t), L is a lag phase or time for the enzyme to start solubilizing the protein substrate, and unsolubleCP is the unsoluble protein content. The variables kd, L, and unsolubleCP are constants. By solving the equation with the measured soluble protein at each time point (solubleCP), the variables kd, L, and unsolubleCP can be predicted and used to calculate solubleCP(t) at any other time. UnsolubleCP can be the remaining residue at 72 h or at the last time point, when protein digestion is substantially exhausted. To optimize the solution for kd across multiple time points, computer software can be used. For example, the software can iteratively solve for combinations of values for kd and L that provide a solubleCP(t) curve with a minimized root mean square error (RMSE) (e.g., a solubleCP(t) curve that touches or comes as close as possible to all the observed solubleCP(t) values).

### EXAMPLES

Various aspects of the present invention can be better understood by reference to the following Examples which are offered by way of illustration. The present invention is not limited to the Examples given herein.

In situ method. In the Examples below, an in situ method of determining rumen protein extent and rate of digestion was performed to compare to the data generated by the in vitro method described herein. The in situ method was performed as follows.

Nylon bags (Ankom Technology) with 45 micron pore size were weighed on a calibrated scale (Sartorius CP 224 S) and the weight of each was recorded. 18 bags were used per cow per feed sample. Each feed ingredient was tested in three animals. 5 grams of air-dried feed sample was weighed and added to each pre-weighed nylon bag. Each bag was sealed twice with an Impulse sealer (AIE-200) by placing the seal near the top side of the nylon bag with 5 mm between the two sealing strips. The top of the bag was cut just above the sealed brim. The nylon bags containing the feed sample were weighed again to record initial ‘gross’ weight. Five bags per feed sample were washed at the same time as the incubated samples in cold tap water and stored in freezer for the initial time point (TO). Three bags per feed per cow were used for the full incubation time of 72 hours. The incubation schedule for each feed sample was 2 bags per cow for each of the following time points—2 hours, 4 hours, 8 hours, 24 hours, and 48 hours (see Table 1).

TABLE 1
Incubation schedule for nylon bags containing a feed sample.

| | | | |
|---|---|---|---|
| Incubation | | | Number |
| hour | Start/In | End/Out | of bags |

| | | | | |
|---|---|---|---|---|
| Day 1 | 72 | h | x | 3 |
| Day 2 | 48 | h | x | 2 |
| Day 3 | 24 | h | x | 2 |

| | | |
|---|---|---|
| Day 4 | 72 h, 48 h, 24 h | x |

| | | | | |
|---|---|---|---|---|
| Day 5 | 8 | h | x | 2 |
| | 4 | h | x | 2 |
| | 2 | h | x | 2 |

| | |
|---|---|
| 8 h, 4 h, 2 h | x |

The bags for each incubation time were placed in mesh laundry washing bags with a weight (RVS, flat/round, 250 g) with one washing bag per timepoint. For each incubation time, a washing bag was placed into the rumen of a fistulated animal at the required day and time, as indicated by an ‘x’ in the Table 1 column labeled ‘Start/In’. At the indicated times the washing bags containing the nylon bags were removed from the rumen and placed in ice water, as indicated by an ‘x’ in the Table 1 column labeled ‘End/Out’. The nylon bags were removed from the washing bag and washed in cold tap water to remove particles and were stored directly in the freezer at −18° C. until analysis.

At the end of the trial, the nylon bags were thawed by au bain marie method in a double boiler water bath at a 50° C. The bags were washed in an automatic washing machine under wool-wash program in cold water without centrifugation. The TO bags were washed separately from the other bags. The washed bags were dried in a dry-oven at 60° C. for a minimum of 48 hours. During drying, the same three bags were weighed at 0, 24, 48, and 52 hours to monitor completion of drying. After drying of bags was complete, bags were removed from the drying oven and weighed immediately to determine final gross weight. Bags were allowed to adapt to room temperature and weighed again. Outliers were identified using the Dixon Q-test (see Robert B. Dean and Wilfrid J. Dixon (1951) “Simplified Statistics for Small Numbers of Observations” Anal. Chem., 1951, 23 (4), 636-638) and removed. Samples were pooled per test feed, animal, and time point in 200 ml cups (urine cups). Samples were stored in closed sample bottles at room temperature until analysis. Percent of undegraded crude protein remaining in the feed samples was determined using total nitrogen analysis via Dumas method (i.e., combusting a sample of known mass in at 800-900° C. in the presence of oxygen) on a Flash2000 (Thermo). Percent crude protein of sample residue was determined by the following equation:

$$Sample CP ( \% ) residue = [ Sample CP g before incubation - sample CP g after incubation - sample CP at T 0 ] / ( sample CP g before incubation )$$

### Example 1. Digestion Assay for Soybean- or Rapeseed-Based Feed Sample

Feed samples having a feed protein equivalent of 0.2 g were weighed into 50 mL conical tubes. The feed protein equivalent is based on crude protein (CP) measurements of the feed sample as determined by NIR analysis. Soybean based feed samples assayed include soybean meal (SBM) of a CP content of 47% and soypass, a rumen bypass soybean product with a CP content of 42%. Rapeseed-containing feed samples assayed include rapeseed meal with a CP content of 35% and rapeseed meal-goldrape with a CP content of 38%.

A borate-phosphate buffer was prepared by weighing 12.2 g of monobasic sodium dihydrogen orthophosphate and 8.91 g of di-sodium tetraborate decahydrate into a beaker and adding distilled water up to 1 L volume. The pH was adjusted to 6.7 with NaOH or HCl, if necessary. The borate-phosphate buffer (40 mL) was added to each tube containing the feed sample. One tube with buffer only and no feed sample was included as a control. The tubes were incubated at 39° C. for 1 hour within an incubator shaking at 90 rpm, with the tubes tilted to a horizontal position to maximize mixing. The 5X protease solution was prepared by weighing the amount of protease from Aspergillus oryzae (Sigma P6110) required for 3.2 U/mL activity into the borate-phosphate buffer. One unit (U) is the amount of enzyme which hydrolyzes 1 mmol of L-leucine-p-nitroanilide per minute, as reported in the Certificate of Analysis provided by the supplier (Sigma-Aldrich). Freshly prepared 5X protease solution (10 mL) was added to each sample including the control with buffer only, resulting in final enzyme activity of 0.64 U/mL in tubes containing 50 mL total volume. The tubes were incubated at 39° C. for 72 hours in an incubator with shaking at 90 rpm, with the tubes tilted to a horizontal position to maximize mixing. Samples of 1 mL supernatant were removed and stored in clean microcentrifuge tubes after 0 h, 4, 24, 48, and 72 h incubation. The supernatant samples were analyzed for soluble protein content by liquid N analyzer or microplate methods, as described in Examples 3 and 4 below.

A pre-calibration step was used to determine the optimal dose of enzyme to obtain rate with highest correlation to in situ degradation curves. Varying concentrations of enzyme were tested with feed samples for which the in-situ degradation curve had been determined. The pre-calibration step may be repeated to ascertain the proper dose to use when new lots of enzyme are received for use in the procedure.

The volume of the samples and amount of feed buffer may be scaled up or down. For example, a scaled down version of the procedure was performed with a total sample volume of 10 mL containing 0.04 g of feed protein equivalent. The ratio of feed protein to buffer volume and the final concentration of enzyme remains unchanged. The results obtained were comparable to data obtained from the 50 mL volume samples described above.

### Example 2. Digestion Assay for Distillers Grain-Based Feed Sample

The feed samples used were distillers dried grains with solubles (DDGS) from wheat with protein contents of 30%, referred to as DDGS, and 28%, referred to as Mix DDGS. The method was the same as described in Example 1 with the exception of the protease solution used. The 5X protease solution used for distillers grain-based ingredients was prepared by weighing the amount of pancreatin from porcine pancreas (Sigma P7545) required to achieve concentration of 0.335 mg/mL in the borate-phosphate buffer. Once added to the tubes, final concentration achieved was 0.067 mg/mL in each tube. The remaining steps of the procedure were carried out as described in Example 1. Samples were analyzed for soluble protein content by liquid N analyzer or microplate methods, as described in Examples 3 and 4 below.

### Example 3. Analysis with Liquid N Analyzer

Total nitrogen of supernatant samples was determined with a Shimadzu Total Organic Carbon Analyzer TOC-L equipped with a TNM-L Total Nitrogen Unit. A 0.5 mL portion of each sample was combined with 10 mL milliQ water to determine mg/mL total nitrogen. A 10 ppm to 250 ppm calibration was used and a 25 ppm check to ensure accuracy. A 21-factor dilution was used to back calculate to determine total mg/mL nitrogen for each sample. Total mg/mL nitrogen of samples was converted to mg/mL soluble protein using conversion factor of 6.25 based on assumption that an average N content of protein is 16%. To account for protein that was solubilized in the initial 1-hour pre-incubation, the soluble protein of the 0-hour sample was subtracted from values of all samples taken at later time points. The “total amount of soluble protein remaining” was determined by subtracting soluble protein at 0 hour from initial crude protein value of 4 mg/mL (0.2 g in 50 mL). Finally, the percent soluble crude protein of the sample was calculated by dividing the soluble protein minus the protein solubilized during the pre-soak by the “total amount of soluble protein remaining” minus the protein solubilized during the pre-soak.

The calculation of the total amount of soluble protein remaining is summarized as follows:
Calculate % soluble crude protein from total nitrogen values:
   Convert total nitrogen to mg/mL soluble protein:

$$mg / L nitrogen \times 6.25 / 1000 = mg / mL soluble protein$$

To account for protein solubilized during presoak before enzyme addition, the value of soluble protein value at 0 hr time point must be subtracted. To calculate the available crude protein after time 0:

$$( 4 mg / mL total theoretical crude protein ) - ( mg / mL protein at time 0 ) = mg / mL crude protein available after time 0 , where total theoretical crude protein = 200 mg CP / 50 mL = 4 mg / mL total crude protein$$

$$To calculate \% soluble protein ( \% sol CP ) : \% sol CP = ( total mg / mL measured - mg / mL time zero ) / ( mg / mL available crude protein at time 0 ) \times 100 \%$$

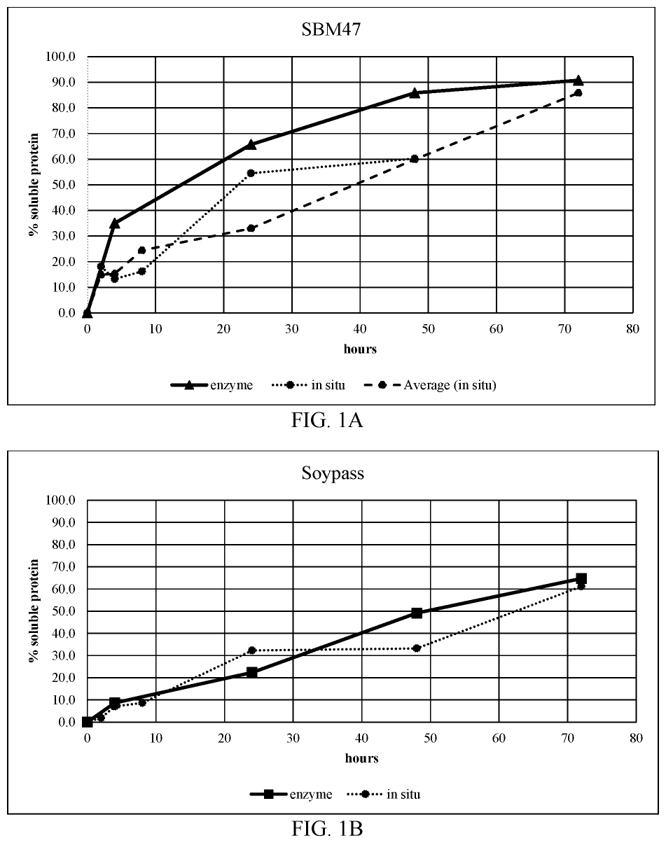

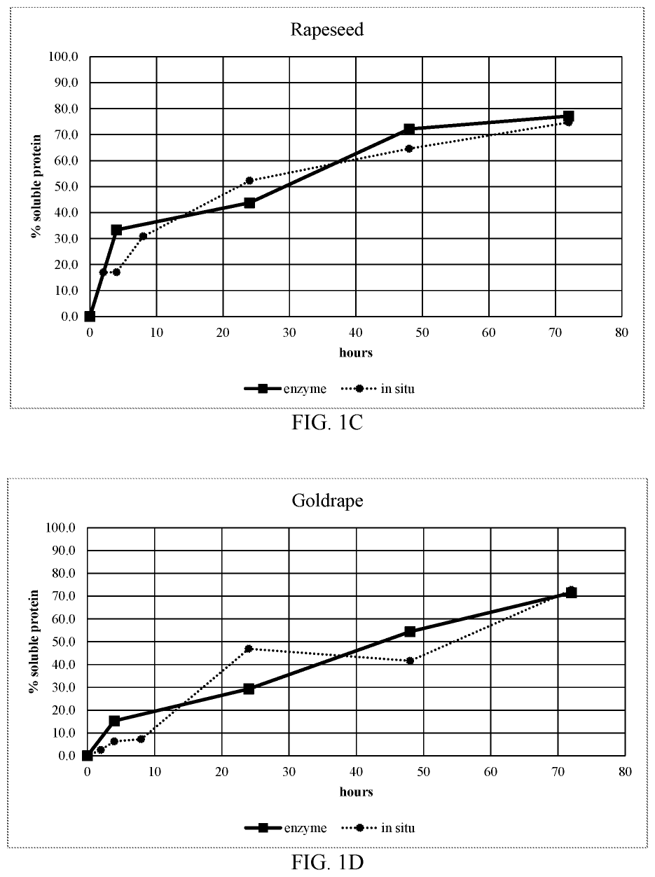

FIGS. 1A-ID illustrate percent soluble protein versus time for Example 1 as determined via the total nitrogen analysis. The data in FIG. 1A for feed sample SBM47 is compared to the most representative dataset obtained using the in situ nylon bag method, labeled ‘in situ’, and the average of all animals, labeled ‘in situ average’, due to an incomplete dataset for the most representative animal. The data in FIGS. 1B-D for feed samples Soypass, Rapeseed, and Goldrape, respectively, is compared to the most representative dataset obtained using the in situ nylon bag method, labeled ‘in situ’. FIGS. 1A-D demonstrate that the digestion assay of Example 1 provided percent soluble protein data that corresponded closely to in situ method data.

### Example 4. Analysis with Microplate Methods

Soluble protein of samples collected from digestion assay were determined via microplate assays. Enzyme activity was stopped by incubation at 99° C. for 5 minutes before proceeding. A Pierce660 protein concentration assay was used to quantify peptides larger than 3 kDa. BSA protein standard was diluted into water to create a standard curve of 0 to 2 mg/mL. 10 μL of either standard or sample were transferred to wells of a 96-well microplate. A multi-channel pipette was used to transfer 150 μL of Pierce660 reagent to each well. The plate was incubated for 5 minutes at room temperature, then absorbance was measured at 660 nm using a spectrophotometer. The BSA standard curve was used to calculate the protein concentration of each sample.

A ninhydrin assay was used to account for the population of free amino acids and small peptides in each sample. Glycine amino acid standard was diluted in water to create a standard curve of 0 to 1000 μg/mL glycine. Other amino acids could be substituted, such as leucine. 15 μL of either standard or sample were transferred to PCR tubes. A multi-channel pipette was used to transfer 135 μL of 2% ninhydrin reagent (Sigma) to each tube. The tubes were heated at 100° C. for 10 minutes in a thermocycler and then cooled for 5 minutes at room temperature. A multi-channel pipette was used to transfer 100 μL of each samples to a 96-well microplate, then absorbance was measured at 570 nm using a spectrophotometer. The glycine standard curve was used to calculate the ug/mL glycine equivalent in each sample. The value for μg/mL glycine equivalent was converted to moles glycine. One mole of glycine was considered to be equal to one mole nitrogen. The moles nitrogen were converted to mg/mL nitrogen. The 6.25 conversion factor was used to convert mg/mL nitrogen to total mg/mL soluble protein for each sample.

The protein concentrations determined by both microplate assays, Pierce660 and ninhydrin, were combined to account for all soluble protein in the samples. It is expected that this method likely overestimates the true value of soluble protein as the two assays will have some amount of overlap. Once the total soluble protein for each sample was calculated, the percent of soluble crude protein was determined by the following. To account for protein that was solubilized in the initial 1-hour pre-incubation, soluble protein at 0 hour was subtracted from values of all samples taken at later time points. ‘Total amount of soluble protein remaining’ was determined by subtracting soluble protein at 0 hour from initial crude protein value of 4 mg/mL (0.2 g in 50 mL). Finally, the percent soluble crude protein of sample was calculated by dividing the soluble protein minus the protein solubilized during the pre-soak by the ‘total amount of soluble protein’ remaining minus the protein solubilized during the pre-soak.

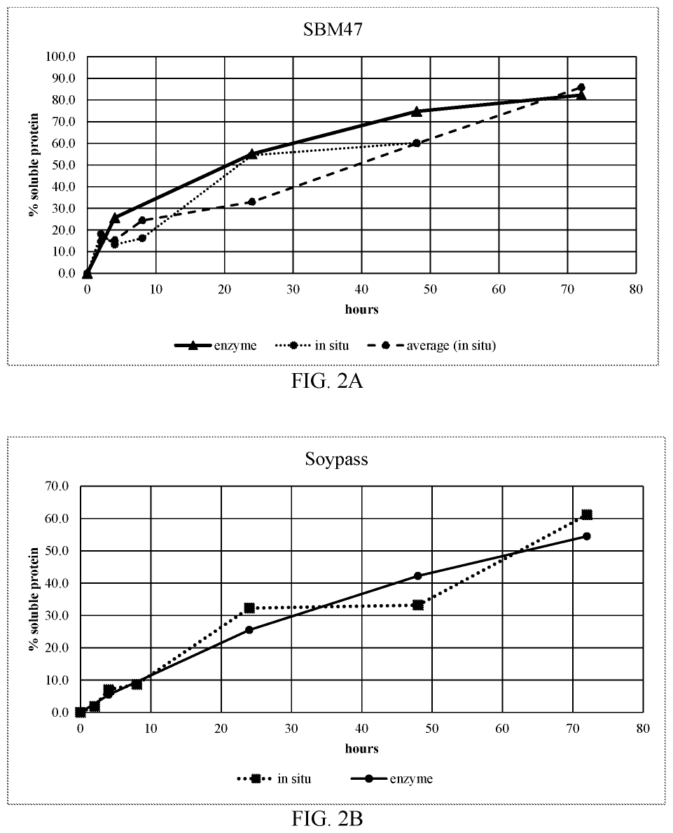

FIGS. 2A-B illustrate percent soluble protein versus time for Example 1 as determined via microplate methods. FIG. 2A for feed sample SBM47 includes a comparison to the most representative dataset obtained using the in situ nylon bag method, labeled ‘in situ’, and the average of all animals ‘in situ average’ due to an incomplete dataset for the most representative animal. FIG. 2B for feed sample Soypass includes a comparison to the most representative dataset obtained using the in situ nylon bag method, labeled ‘in situ’. FIGS. 2A-B demonstrate that the digestion assay of Example 1 provided percent soluble protein data that corresponded closely to in situ method data.

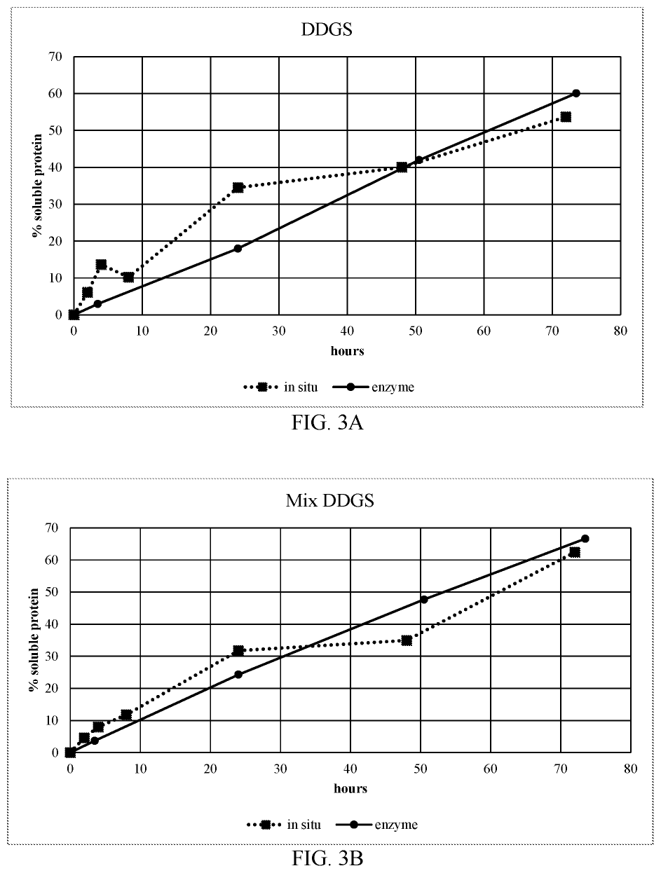

FIGS. 3A-B illustrate percent soluble protein versus time for Example 2 as determined via microplate methods. FIG. 3A for feed sample DDGS, and FIG. 3B for feed sample “Mix DDGS”, both include a comparison to most representative dataset obtained using the in situ nylon bag method, labeled “in situ”. FIGS. 3A-B demonstrate that the digestion assay of Example 2 provided percent soluble protein data that corresponded closely to in situ method data.

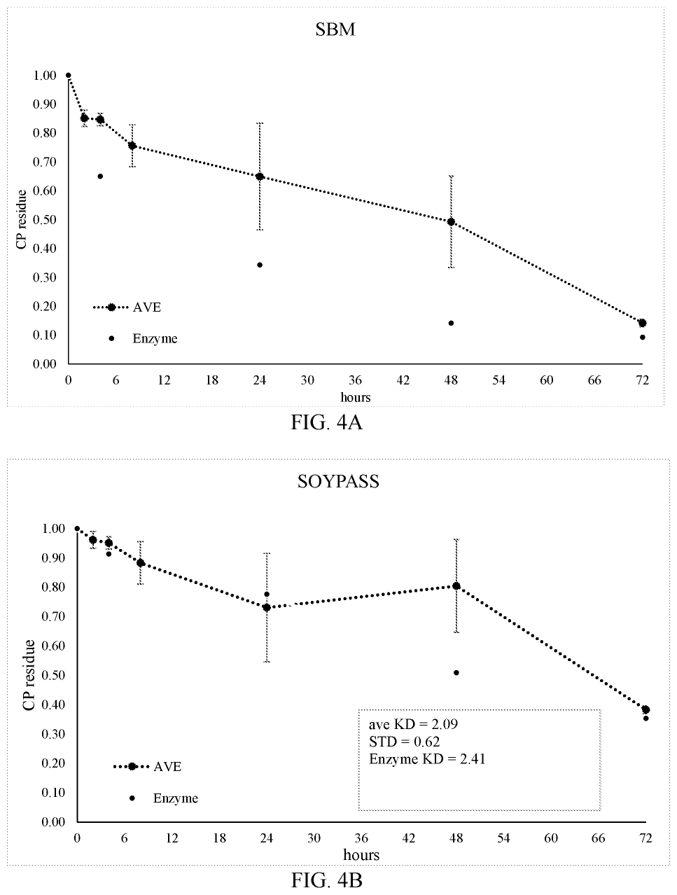

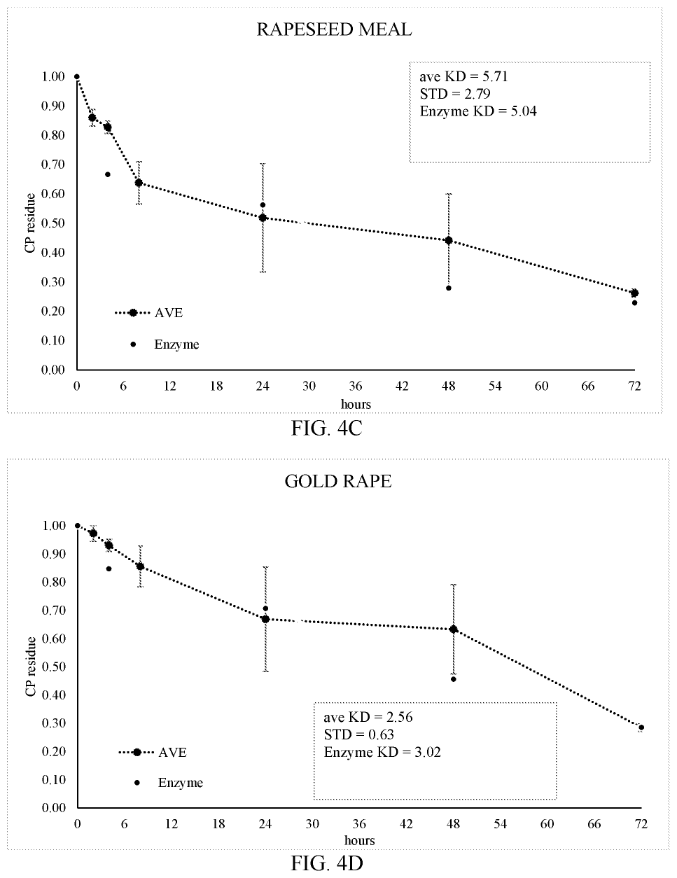

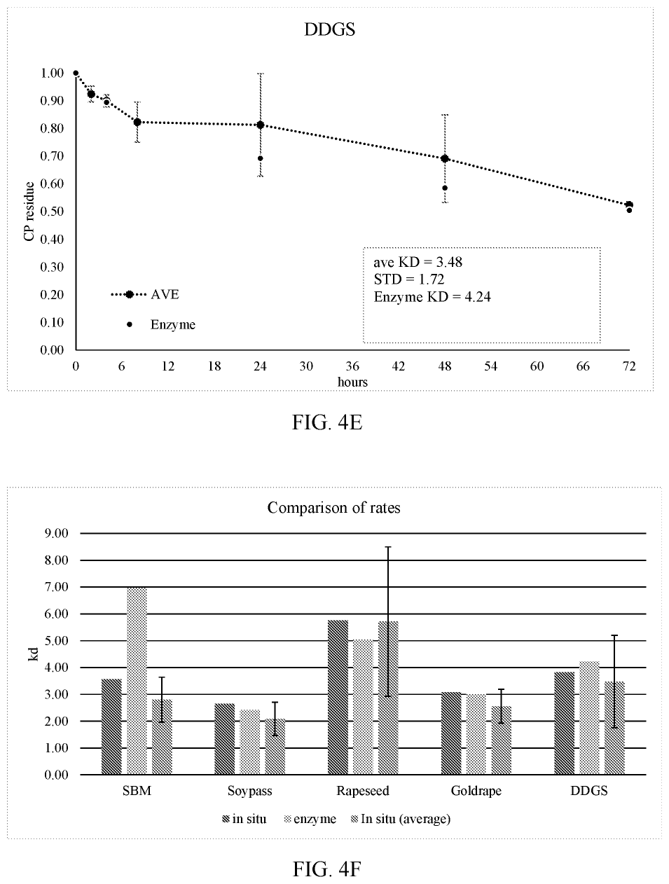

FIG. 4A illustrates a plot of the proportion of remaining crude protein in the residue versus time for SBM47 in Example 1. FIG. 4B illustrates a plot of the proportion of remaining crude protein in the residue versus time for soypass in Example 1. FIG. 4C illustrates a plot of the proportion of remaining crude protein in the residue versus time for rapeseed meal in Example 1. FIG. 4D illustrates a plot of the proportion of remaining crude protein in the residue versus time for goldrape in Example 1. FIG. 4E illustrates a plot of the proportion of remaining crude protein in the residue versus time for DDGS in Example 2. FIG. 4F illustrates a comparison of rates for SBM, soypass, rapeseed, goldrape, from Example 1, and DDGS, from Example 2. FIG. 4F illustrates that the determined rates were within 1 standard deviation for all samples other than SBM47, demonstrating close correspondence with in situ method data.

### Example 5. Digestion Assay for Legume Hay or Haylage Feed Sample

Feed samples having a feed protein equivalent of 0.2 g were weighed into 50 mL conical tubes. The feed protein equivalent is based on crude protein (CP) measurements of the feed sample as determined by NIR analysis. The hay feed samples assayed include a legume hay (HL) of a CP content of 21%, a legume hay (HL) of a CP content of 17%, and legume hay (HL) of a CP content of 15%. The haylage feed sample assayed include alfalfa haylage (EHL) with a CP content of 24%.

A borate-phosphate buffer was prepared by weighing 12.2 g of monobasic sodium dihydrogen orthophosphate and 8.91 g of di-sodium tetraborate decahydrate into a beaker and adding distilled water up to 1 L volume. The pH was adjusted to 6.7 with NaOH or HCl, if necessary. The borate-phosphate buffer (40 mL) was added to each tube containing the feed sample. The tubes were incubated at 39° C. for 1 hour within an incubator shaking at 90 rpm, with the tubes tilted to a horizontal position to maximize mixing. The 5X protease solution was prepared by weighing the amount of protease from Aspergillus oryzae (Sigma P6110) required for 3.2 U/mL activity into the borate-phosphate buffer. One unit (U) is the amount of enzyme which hydrolyzes 1 mmol of L-leucine-p-nitroanilide per minute, as reported in the Certificate of Analysis provided by the supplier (Sigma-Aldrich). Freshly prepared 5X protease solution (10 mL) was added to each sample including the control with buffer only, resulting in final enzyme activity of 0.64 U/mL in tubes containing 50 mL total volume. The tubes were incubated at 39° C. for 72 hours in an incubator with shaking at 90 rpm, with the tubes tilted to a horizontal position to maximize mixing. Samples of 1 mL supernatant were removed and stored in clean microcentrifuge tubes after 0 h, 4, 24, 48, and 72 h incubation. The supernatant samples were analyzed for soluble protein content by liquid N analyzer, as described below.

Analysis with Liquid N Analyzer

Total nitrogen of supernatant samples was determined with a Shimadzu Total Organic Carbon Analyzer TOC-L equipped with a TNM-L Total Nitrogen Unit. A 0.5 mL portion of each sample was combined with 10 mL milliQ water to determine mg/mL total nitrogen. A 10 ppm to 250 ppm calibration was used and a 25 ppm check to ensure accuracy. A 21-factor dilution was used to back calculate to determine total mg/mL nitrogen for each sample. Total mg/mL nitrogen of samples was converted to mg/mL soluble protein using conversion factor of 6.25 based on assumption that an average N content of protein is 16%. To account for protein that was solubilized in the initial 1-hour pre-incubation, the soluble protein of the 0-hour sample was subtracted from values of all samples taken at later time points. The ‘total amount of soluble protein remaining’ was determined by subtracting soluble protein at 0 hour from initial crude protein value of 4 mg/mL (0.2 g in 50 mL). Finally, the percent soluble crude protein of the sample was calculated by dividing the soluble protein minus the protein solubilized during the pre-soak by the ‘total amount of soluble protein remaining’ minus the protein solubilized during the pre-soak.

The calculation of the total amount of soluble protein remaining is summarized as follows:
Calculate % soluble crude protein from total nitrogen values:
   Convert total nitrogen to mg/mL soluble protein:

$$mg / L nitrogen \times 6.25 / 1000 = mg / mL soluble protein$$

To account for protein solubilized during presoak before enzyme addition, the value of soluble protein value at 0 hr time point must be subtracted. To calculate the available crude protein after time 0:

$$( 4 mg / mL total theoretical crude protein ) - ( mg / mL protein at time 0 ) = mg / mL crude protein available after time 0 , where total theoretical crude protein = 200 mg CP / 50 mL = 4 mg / mL total crude protein$$

$$To calculate \% soluble protein ( \% sol CP ) : \% sol CP = ( total mg / mL measured - mg / mL time zero ) / ( mg / mL available crude protein at time 0 ) \times 100 \%$$

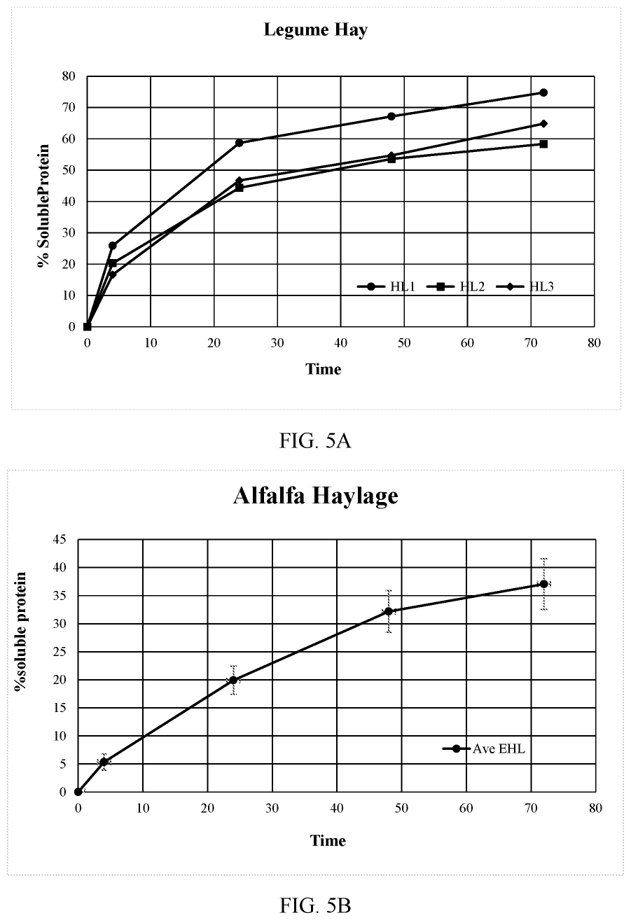

The data in FIG. 5A for feed samples Legume Hay compare the most representative dataset obtained using legume hay of 3 different CP amounts (21%,17% and 15%), labeled HL1, HL2, and HL3. The data in FIG. 5B illustrate average of percent of soluble protein versus time for feed sample alfalfa haylage.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects

The following exemplary aspects are provided, the numbering of which is not to be construed as designating levels of importance:

Para. A provides an in vitro method of predicting rumen digestible protein of a feed sample, the method comprising:
forming an aqueous test composition comprising
   the feed sample,
   a buffer, and
   one or more digestive enzymes;
incubating the test composition;
removing one or more test samples of the test composition at time points during the incubating, the time points comprising about 0 h and at least one time point after about 0 h;
measuring soluble protein content of the one or more test samples; and
using the measured soluble protein content of the one or more test samples to predict a rate and/or extent of rumen protein digestion of the feed sample.

Para. B provides the method of para. A, wherein the feed sample comprises an animal feed, a feedstuff, or a combination thereof.

Para. C provides the method of para. B, wherein the animal feed comprises a complete feed, a premix, a concentrate, a base mix, a supplement, a top dress, or a combination thereof.

Para. D provides the method of any one of paras. A-C, wherein the feed sample comprises barley, blood meal, bone meal, Brewer's grain, corn grain, corn gluten meal, corn gluten feed, cottonseed (e.g., whole or meal), distiller's grain, fish meal, hominy, feather meal, molasses, peanut skins, soybeans (e.g., whole or meal), tallow, wheat (e.g., whole, bran or middlings), a rumen-protected amino acid, a byproduct thereof, or a combination thereof.

Para. E provides the method of any one of paras. A-D, wherein the feed sample comprises forage, corn, wheat, soybean, oats, barley, beet pulp, citrus pulp, cottonseed, sunflowers, canola/rapeseed, rice, peas, rye, distillers grains, a byproduct thereof, or a combination thereof.

Para. F provides the method of any one of paras. A-E, wherein the feed sample comprises distillers grains, soybeans, rapeseed, a byproduct thereof, or a combination thereof.

Para. G provides the method of any one of paras. A-F, wherein the feed sample comprises soybeans, rapeseed, byproducts thereof, or a combination thereof.

Para. H provides the method of any one of paras. A-G, wherein the feed sample comprises distillers grains, byproducts thereof, or a combination thereof.

Para. I provides the method of any one of paras. A-H, wherein forming the test composition comprises combining the feed sample and the buffer to form a pre-test composition, and pre-incubating the pre-test composition prior to adding the one or more digestive enzymes to form the aqueous test composition.

Para. J provides the method of para. I, wherein the pre-incubating of the pre-test composition comprises agitation, elevated temperature, or a combination thereof.

Para. K provides the method of para. J, wherein the agitation comprises agitation at about 50 RPM to about 150 RPM.

Para. L provides the method of any one of paras. J-K, wherein the agitation comprises agitation at about 80 RPM to about 100 RPM.

Para. M provides the method of any one of paras. I-L, wherein the pre-incubating of the pre-test composition comprises agitation at about 50 RPM to about 150 RPM at a temperature of about 35° C. to about 45° C. for about 30 minutes to about 2 hours.

Para. N provides the method of any one of paras. I-M, wherein the pre-incubating of the pre-test composition comprises agitation at a temperature of about 38° C. to about 40° C. for about 45 minutes to about 1.5 hours.

Para. O provides the method of any one of paras. I-N, wherein the pre-incubating of the pre-test composition comprises agitation at a temperature of about 39° C. for about 1 hour.

Para. P provides the method of any one of paras. A-O, wherein the buffer has a pH of about 5 to about 9.

Para. Q provides the method of any one of paras. A-P, wherein the buffer has a pH of about 5.8 to about 7.2.

Para. R provides the method of any one of paras. A-Q, wherein the buffer has a pH of about 6.7.

Para. S provides the method of any one of paras. A-R, wherein the test composition has a pH of about 5 to about 9.

Para. T provides the method of any one of paras. A-S, wherein the test composition has a pH of about 5.8 to about 7.2.

Para. U provides the method of any one of paras. A-T, wherein the test composition has a pH of about 6.7.

Para. V provides the method of any one of paras. A-U, wherein the buffer comprises citric acid, acetic acid, KH₂PO₄, K₂HPO₄, K₃PO₄, HCl, KCl, N-cyclohexyl-2-aminoethanesulfonic acid (CHES), glycine, potassium hydrogen phthalate, tetraboric acid, boric acid, [tris(hydroxymethyl)methylamino]propanesulfonic acid (TAPS), 2-(bis(2-hydroxyethyl)amino)acetic acid (bicine), tris(hydroxylmethyl)aminomethane (tris), N-[tris(hydroxymethyl)methyl]glycine (tricine), 3-[N-tris(hydroxymethyl)methylamino]-2-hydroxypropanesulfonic acid (TAPSO), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), piperazine-N,N′-bis(2-ethanesulfonic acid) (PIPES), dimethylarsenic acid (cacodylate), 2-(N-morpholino)ethanesulfonic acid (MES), a salt thereof, borate, sodium hydroxide, potassium hydroxide, sodium tetraborate, sodium carbonate, potassium carbonate, potassium chloride, sodium chloride, a sodium phosphate, phosphate buffered saline (PBS), hydrates thereof, or a combination thereof.

Para. W provides the method of any one of paras. A-V, wherein the buffer comprises a sodium phosphate, phosphate buffered saline (PBS), a borate-phosphate buffer, or a combination thereof.

Para. X provides the method of any one of paras. A-W, wherein the buffer comprises monobasic sodium dihydrogen orthophosphate and di-sodium tetraborate decahydrate.

Para. Y provides the method of any one of paras. A-X, wherein the one or more digestive enzymes comprise a protease, a metalloprotease, a lyase, an amylase, or a combination thereof.

Para. Z provides the method of any one of paras. A-Y, wherein the one or more digestive enzymes comprise a bacterial protease, an animal protease, a virus protease, a plant protease, a fungal protease, a serine protease, a cysteine protease, a threonine protease, an aspartic protease, a glutamic protease, a metalloprotease, an asparagine peptide lyase, an amylase derives from a pancreas, or a combination thereof.

Para. AA provides the method of any one of paras. A-Z, wherein the one or more digestive enzymes comprise a mammalian trypsin, trypsin from porcine pancreas, one or more Aspergillus-derived digestive enzymes, one or more proteases from Aspergillus oryzae, pancreatin from porcine pancreas, an amylase derived from a porcine pancreas, a protease derived from Bacillus licheniformis, a protease derived from Bacillus amyloliquefaciens, or a combination thereof.

Para. AB provides the method of any one of paras. A-AA, wherein the one or more digestive enzymes comprise one or more proteases from Aspergillus oryzae, pancreatin from porcine pancreas, or a combination thereof.

Para. AC provides the method of any one of paras. A-AB, wherein the one or more digestive enzymes and a concentration thereof in the test composition is sufficient such that a rate of formation of solubilized protein in the test composition during the incubation is within about 20% of a rate of protein digestion of the feed sample in a rumen stomach, and wherein the digestive enzyme and a concentration thereof in the test composition is sufficient such that an extent of formation of solubilized protein in the test composition during the incubation is within about 10% of an extent of protein digestion of the feed sample in a rumen stomach.

Para. AD provides the method of any one of paras. A-AC, wherein the one or more digestive enzymes and a concentration thereof in the test composition is sufficient such that a rate and extent of formation of solubilized protein in the test composition during the incubation is within about 10% of a rate and extent of protein digestion of the feed sample in a rumen stomach.

Para. AE provides the method of any one of paras. A-AD, wherein the one or more digestive enzymes and a concentration thereof in the test composition is sufficient such that a rate and extent of formation of solubilized protein in the test composition during the incubation is within about 5% of a rate and extent of protein digestion of the feed sample in a rumen stomach.

Para. AF provides the method of any one of paras. A-AE, wherein the one or more digestive enzymes and a concentration thereof in the test composition is sufficient such that a rate and extent of formation of solubilized protein in the test composition during the incubation is within about 3 standard deviations of a rate and extent of protein digestion of the feed sample in a rumen stomach.

Para. AG provides the method of any one of paras. A-AF, wherein the one or more digestive enzymes and a concentration thereof in the test composition is sufficient such that a rate and extent of formation of solubilized protein in the test composition during the incubation is within about 2 standard deviations of a rate and extent of protein digestion of the feed sample in a rumen stomach.

Para. AI provides the method of any one of paras. A-AG, wherein the one or more digestive enzymes comprise one or more proteases from Aspergillus oryzae.

Para. AJ provides the method of para. AI, wherein the one or more proteases from Aspergillus oryzae have an activity of about 0.1 U/mL to about 1.5 U/mL in the test composition per about 0.2 g of protein in the feed sample.

Para. AK provides the method of any one of paras. AI-AJ, wherein the one or more proteases from Aspergillus oryzae have an activity of about 0.4 U/mL to about 0.9 U/mL in the test composition per about 0.2 g of protein in the feed sample.

Para. AL provides the method of any one of paras. AI-AK, wherein the one or more proteases from Aspergillus oryzae have an activity of about 0.64 U/mL in the test composition per about 0.2 g of protein in the feed sample.

Para. AM provides the method of any one of paras. AI-AL, wherein the feed sample comprises soybeans, rapeseed, byproducts thereof, or a combination thereof.

Para. AN provides the method of any one of paras. A-AM, wherein the one or more digestive enzymes comprise protease from porcine pancreas.

Para. AO provides the method of any one of paras. A-AN, wherein the one or more digestive enzymes comprise one or more enzymes from porcine pancreas.

Para. AP provides the method of para. AN, wherein the one or more enzymes from porcine pancreas have a concentration of about 0.01 mg/mL to about 0.15 mg/mL in the test composition per about 0.2 g of protein in the feed sample.

Para. AQ provides the method of any one of paras. AN-AP, wherein the one or more enzymes from porcine pancreas have a concentration of about 0.04 mg/mL to about 0.09 mg/mL in the test composition per about 0.2 g of protein in the feed sample.

Para. AR provides the method of any one of paras. AN-AQ, wherein the one or more enzymes from porcine pancreas have a concentration of about 0.067 mg/mL in the test composition per about 0.2 g of protein in the feed sample.

Para. AS provides the method of any one of paras. AN-AR, wherein the feed sample comprises distillers grains, byproducts thereof, or a combination thereof.

Para. AT provides the method of any one of paras. A-AS, further comprising pre-determining an amount or activity level of the one or more digestive enzymes in the test composition for the feed sample.

Para. AU provides the method of para. AT, wherein the pre-determining of the amount or the activity level of the one or more digestive enzymes in the test composition comprises optimizing an amount of the one or more digestive enzymes used in the test composition such that a rate and extent of formation of solubilized protein in the test composition during the incubation is within about 3 standard deviations of a rate and extent of protein digestion of the feed sample in a rumen stomach.

Para. AV provides the method of any one of paras. AT-AU, wherein the pre-determining of the amount or the activity level of the one or more digestive enzymes in the test composition comprises optimizing an amount of the one or more digestive enzymes used in the test composition such that a rate and extent of formation of solubilized protein in the test composition during the incubation is within about 10% of a rate and extent of protein digestion of the feed sample in a rumen stomach.

Para. AW provides the method of any one of paras. A-AV, wherein the incubating is performed at a temperature of about 30° C. to about 50° C.

Para. AX provides the method of any one of paras. A-AW, wherein the incubating is performed at a temperature of about 35° C. to about 45° C.

Para. AY provides the method of any one of paras. A-AX, wherein the incubating is performed at a temperature of about 38° C. to about 40° C.

Para. AZ provides the method of any one of paras. A-AY, wherein the incubating is performed at a temperature of about 39° C.

Para. BA provides the method of any one of paras. A-AZ, wherein the incubating is performed for a duration of about 1 minute to about 1 week.

Para. BB provides the method of any one of paras. A-BA, wherein the incubating is performed for a duration of about 30 minutes to about 96 hours.

Para. BC provides the method of any one of paras. A-BB, wherein the incubating is performed for a duration of about 1 hour to about 72 hours.

Para. BD provides the method of any one of paras. A-BC, wherein the incubating comprises agitating the test composition.

Para. BE provides the method of para. BD, wherein the agitation comprises agitation at about 50 RPM to about 150 RPM.

Para. BF provides the method of any one of paras. BD-BE, wherein the agitation comprises agitation at about 80 RPM to about 100 RPM.

Para. BG provides the method of any one of paras. A-BF, wherein the incubating is performed with agitation at about 50 RPM to about 150 RPM at a temperature of about 35° C. to about 45° C. for about 1 hour to about 72 hours.

Para. BH provides the method of any one of paras. A-BG, wherein the incubating is performed with agitation at about 80 RPM to about 100 RPM at about 39° C. for about 1 hour to about 72 hours.

Para. BI provides the method of any one of paras. A-BH, wherein the removing of the sample of the test composition comprises removing a portion of supernatant solution from the test composition.

Para. BJ provides the method of any one of paras. A-BI, wherein the time points at which the sample of the test composition is removed during the incubating comprises about 0 h and at least two time points after about 0 h.

Para. BK provides the method of any one of paras. A-BJ, wherein the time points at which the sample of the test composition is removed during the incubating comprises about 0 h and at least three time points after about 0 h.

Para. BL provides the method of any one of paras. A-BK, wherein the at least one time point after about 0 h at which the sample of the test composition is removed during the incubating comprises one or more of about 4 h, 24, 48, and about 72 h.

Para. BM provides the method of any one of paras. A-BL, wherein the measuring of the soluble protein content of the one or more test samples comprises a combustion method (e.g., on a LECO instrument), a digestion method, total nitrogen analysis, a microplate assay method, a cuvette assay method (e.g., using the same reagents as a microplate assay method), a 2,4,6-trinitrobenzene sulfonic acid (TNBS) assay, a bicinchoninic acid (BCA) assay, acid hydrolysis followed by amino acid measurement (e.g., via HPLC or free amine detection reagents such as ninhydrin or TNBS), amino acid HPLC, quantitative protein gel, microfluidic electrophoretic separation (e.g., using a LapChip system), Kjeldahl method, Lowry colorimetric method, or a combination thereof.

Para. BN provides the method of any one of paras. A-BM, wherein the measuring of the soluble protein content comprises total nitrogen analysis and/or a microplate assay method.

Para. BO provides the method of any one of paras. A-BN, wherein the measuring of the soluble protein content comprises total nitrogen analysis.

Para. BP provides the method of any one of paras. A-BO, wherein the measuring of the soluble protein content comprises a microplate assay method.

Para. BQ provides the method of para. BP, wherein the microplate assay method comprises a protein concentration assay, an assay for detection of free amino groups, an assay for colorimetric detection of free amino groups, or a combination thereof.

Para. BR provides the method of any one of paras. BP-BQ, wherein the microplate assay method comprises a ninhydrin assay.

Para. BS provides the method of para. BR, wherein the measuring of the soluble protein content comprises combining a protein concentration determined via a protein concentration assay and a protein concentration determined via a ninhydrin assay to determine the measured soluble protein content of the sample.

Para. BT provides the method of any one of paras. A-BS, wherein the method comprises using the measured soluble protein content of the one or more test samples to predict a rate of rumen protein digestion of the feed sample.

Para. BU provides the method of any one of paras. A-BT, wherein the method comprises using the measured soluble protein content of the one or more test samples to predict an extent of rumen protein digestion of the feed sample.

Para. BV provides the method of any one of paras. A-BU, wherein the method comprises using the measured soluble protein content of the one or more test samples to predict an extent and rate of rumen protein digestion of the feed sample.

Para. BW provides the method of any one of paras. A-BV, wherein the using of the measured soluble protein content of the one or more test samples to predict an extent of rumen protein digestion of the feed sample comprises subtracting the measured soluble protein content of the test composition at about 0 h from the measured soluble protein content of the test composition at the least one time point after about 0 h.

Para. BX provides the method of any one of paras. A-BW, wherein the using of the measured soluble protein content of the one or more test samples to predict a rate of rumen protein digestion of the feed sample comprises solving for kd in the equation:

$$soluble CP ( t ) = {soluble CP}^{( - kd * ( t - L ) )} unsoluble CP$$

wherein
   solubleCP(t) is the protein content at time t,
   kd is the predicted rate of rumen protein digestion of the feed sample,
   t is a time the samples were removed,
   solubleCP is the measured soluble protein content of the removed sample at time t,
   L is a lag phase or time for the enzyme to start solubilizing the protein substrate, and
   unsolubleCP is the unsoluble protein content.

Para. BY provides an in vitro method of predicting rumen digestible protein of a feed sample, the method comprising:
forming an aqueous test composition comprising
   the feed sample,
   a buffer having a pH of about 5 to about 9, and
   one or more digestive enzymes, wherein forming the test composition comprises combining the feed sample and the buffer to form a pre-test composition, and pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition;
incubating the test composition at a temperature of about 35° C. to about 45° C. with agitation at about 50 RPM to about 150 RPM;
removing one or more test samples of the test composition at time points during the incubating, the time points comprising about 0 h and at least one time point after about 0 h;
measuring soluble protein content of the one or more test samples; and
using the measured soluble protein content of the one or more test samples to predict a rate and/or extent of rumen protein digestion of the feed sample.

Para. BZ provides an in vitro method of predicting rumen digestible protein of a feed sample, the method comprising:
forming an aqueous test composition comprising
   the feed sample, wherein the feed sample comprises soybeans and/or rapeseed,
   a buffer having a pH of about 5.8 to about 7.2, and
   one or more digestive enzymes, wherein forming the test composition comprises combining the feed sample and the buffer to form a pre-test composition, and pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition, the one or more digestive enzymes comprising one or more proteases from Aspergillus oryzae having an activity of about 0.1 U/mL to about 1.5 U/mL in the test composition per about 0.2 g of protein in the feed sample;
incubating the test composition at a temperature of about 35° C. to about 45° C. with agitation at about 50 RPM to about 150 RPM;
removing one or more test samples of the test composition at time points during the incubating, the time points comprising about 0 h and at least one time point after about 0 h;
measuring soluble protein content of the one or more test samples; and
using the measured soluble protein content of the one or more test samples to predict a rate and/or extent of rumen protein digestion of the feed sample.

Para. CA provides an in vitro method of predicting rumen digestible protein of a feed sample, the method comprising:
forming an aqueous test composition comprising
   the feed sample, wherein the feed sample comprises distillers grains,
   a buffer having a pH of about 5.8 to about 7.2, and
   one or more digestive enzymes, wherein forming the test composition comprises combining the feed sample and the buffer to form a pre-test composition, and pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition, the one or more digestive enzymes comprising pancreatin from porcine pancreas having a concentration of about 0.01 mg/mL to about 0.15 mg/mL in the test composition per about 0.2 g of protein in the feed sample;
incubating the test composition at a temperature of about 35° C. to about 45° C. with agitation at about 50 RPM to about 150 RPM;
removing one or more test samples of the test composition at time points during the incubating, the time points comprising about 0 h and at least one time point after about 0 h;
measuring soluble protein content of the one or more test samples; and
using the measured soluble protein content of the one or more test samples to predict a rate and/or extent of rumen protein digestion of the feed sample.

Para. CB provides the method of any one or any combination of paras. A-CA optionally configured such that all elements or options recited are available to use or select from.

What is claimed is:

1. An in vitro method of predicting rumen digestible protein of a feed sample, the method comprising:
forming an aqueous test composition comprising
the feed sample,
a buffer, and
one or more digestive enzymes;
incubating the test composition;
removing one or more test samples of the test composition at time points during the incubating, the time points comprising about 0 h and at least one time point after about 0 h;
measuring soluble protein content of the one or more test samples; and
using the measured soluble protein content of the one or more test samples to predict a rate and/or extent of rumen protein digestion of the feed sample.

2. The method of claim 1, wherein the feed sample comprises barley, blood meal, bone meal, Brewer's grain, corn grain, corn gluten meal, corn gluten feed, cottonseed, distiller's grain, fish meal, hominy, feather meal, molasses, peanut skins, soybeans, tallow, wheat, a rumen-protected amino acid, forage, corn, wheat, soybean, oats, barley, beet pulp, citrus pulp, cottonseed, sunflowers, canola/rapeseed, rice, peas, rye, distillers grains, a byproduct thereof, or a combination thereof.

3. The method of claim 1, wherein the feed sample comprises distillers grains, soybeans, rapeseed, a byproduct thereof, or a combination thereof.

4. The method of claim 1, wherein forming the test composition comprises combining the feed sample and the buffer to form a pre-test composition, and pre-incubating the pre-test composition prior to adding the one or more digestive enzymes to form the aqueous test composition.

5. The method of claim 4, wherein the pre-incubating of the pre-test composition comprises agitation at about 50 RPM to about 150 RPM at a temperature of about 35° C. to about 45° C. for about 30 minutes to about 2 hours.

6. The method of claim 1, wherein the buffer has a pH of about 5 to about 9, and wherein the test composition has a pH of about 5 to about 9.

7. The method of claim 1, wherein the buffer comprises monobasic sodium dihydrogen orthophosphate and di-sodium tetraborate decahydrate.

8. The method of claim 1, wherein the one or more digestive enzymes comprise a protease, a metalloprotease, a lyase, an amylase, or a combination thereof.

9. The method of claim 1, wherein the one or more digestive enzymes comprise a bacterial protease, an animal protease, a virus protease, a plant protease, a fungal protease, a serine protease, a cysteine protease, a threonine protease, an aspartic protease, a glutamic protease, a metalloprotease, an asparagine peptide lyase, an amylase derives from a pancreas, or a combination thereof.

10. The method of claim 1, wherein the one or more digestive enzymes comprise one or more proteases from Aspergillus oryzae, pancreatin from porcine pancreas, or a combination thereof.

11. The method of claim 1, wherein the one or more digestive enzymes and a concentration thereof in the test composition is sufficient such that:
a rate of formation of solubilized protein in the test composition during the incubation is within about 20% of a rate of protein digestion of the feed sample in a rumen stomach, and wherein the digestive enzyme and a concentration thereof in the test composition is sufficient such that an extent of formation of solubilized protein in the test composition during the incubation is within about 10% of an extent of protein digestion of the feed sample in a rumen stomach, or
a rate and extent of formation of solubilized protein in the test composition during the incubation is within about 1 standard deviations of a rate and extent of protein digestion of the feed sample in a rumen stomach, or
a combination thereof.

12. The method of claim 1, wherein the one or more digestive enzymes comprise one or more proteases from Aspergillus oryzae, wherein the one or more proteases from Aspergillus oryzae have an activity of about 0.1 U/mL to about 1.5 U/mL in the test composition per about 0.2 g of protein in the feed sample, and wherein the feed sample comprises soybeans, rapeseed, byproducts thereof, or a combination thereof.

13. The method of claim 1, wherein the one or more digestive enzymes comprise protease from porcine pancreas, wherein the one or more enzymes from porcine pancreas have a concentration of about 0.01 mg/mL to about 0.15 mg/mL in the test composition per about 0.2 g of protein in the feed sample, and wherein the feed sample comprises distillers grains, byproducts thereof, or a combination thereof.

14. The method of claim 1, further comprising pre-determining an amount or activity level of the one or more digestive enzymes in the test composition for the feed sample, the pre-determining comprising:
optimizing an amount of the one or more digestive enzymes used in the test composition such that a rate and extent of formation of solubilized protein in the test composition during the incubation is within about 1 standard deviation of a rate and extent of protein digestion of the feed sample in a rumen stomach, or
optimizing an amount of the one or more digestive enzymes used in the test composition such that a rate and extent of formation of solubilized protein in the test composition during the incubation is within about 20% of a rate and extent of protein digestion of the feed sample in a rumen stomach, or
a combination thereof.

15. The method of claim 1, wherein the incubating is performed with agitation at about 50 RPM to about 150 RPM at a temperature of about 35° C. to about 45° C. for about 1 hour to about 72 hours.

16. The method of claim 1, wherein the method comprises using the measured soluble protein content of the one or more test samples to predict a rate of rumen protein digestion of the feed sample.

17. The method of claim 1, wherein the method comprises using the measured soluble protein content of the one or more test samples to predict an extent of rumen protein digestion of the feed sample.

18. The method of claim 1, wherein the using of the measured soluble protein content of the one or more test samples to predict a rate of rumen protein digestion of the feed sample comprises solving for kd in the equation:
$$soluble CP ( t ) = {soluble CP}^{( - kd * ( t - L ) )} unsoluble CP$$
wherein
solubleCP(t) is the protein content at time t,
kd is the predicted rate of rumen protein digestion of the feed sample,
t is a time the samples were removed,
solubleCP is the measured soluble protein content of the removed sample at time t,
L is a lag phase or time for the enzyme to start solubilizing the protein substrate, and
unsolubleCP is the unsoluble protein content.

19. An in vitro method of predicting rumen digestible protein of a feed sample, the method comprising:
forming an aqueous test composition comprising
the feed sample, wherein the feed sample comprises soybeans and/or rapeseed,
a buffer having a pH of about 5.8 to about 7.2, and
one or more digestive enzymes, wherein forming the test composition comprises combining the feed sample and the buffer to form a pre-test composition, and pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition, the one or more digestive enzymes comprising one or more proteases from Aspergillus oryzae having an activity of about 0.1 U/mL to about 1.5 U/mL in the test composition per about 0.2 g of protein in the feed sample;
incubating the test composition at a temperature of about 35° C. to about 45° C. with agitation at about 50 RPM to about 150 RPM;
removing one or more test samples of the test composition at time points during the incubating, the time points comprising about 0 h and at least one time point after about 0 h;
measuring soluble protein content of the one or more test samples; and
using the measured soluble protein content of the one or more test samples to predict a rate and/or extent of rumen protein digestion of the feed sample.

20. An in vitro method of predicting rumen digestible protein of a feed sample, the method comprising:
forming an aqueous test composition comprising
the feed sample, wherein the feed sample comprises distillers grains,
a buffer having a pH of about 5.8 to about 7.2, and
one or more digestive enzymes, wherein forming the test composition comprises combining the feed sample and the buffer to form a pre-test composition, and pre-incubating the pre-test composition with agitation at about 50 RPM to about 150 RPM at a temperature of about 35° C. to about 45° C. for about 30 minutes to about 2 hours prior to adding the one or more digestive enzymes to form the aqueous test composition, the one or more digestive enzymes comprising pancreatin from porcine pancreas having a concentration of about 0.01 mg/mL to about 0.15 mg/mL in the test composition per about 0.2 g of protein in the feed sample;
incubating the test composition at a temperature of about 35° C. to about 45° C. with agitation at about 50 RPM to about 150 RPM;
removing one or more test samples of the test composition at time points during the incubating, the time points comprising about 0 h and at least one time point after about 0 h;
measuring soluble protein content of the one or more test samples; and
using the measured soluble protein content of the one or more test samples to predict a rate and/or extent of rumen protein digestion of the feed sample.
